(19) United States
(12) Patent Application Publication
(10) Pub. No.: US 2008/0293689 A1
(43) Pub. Date: Nov. 27, 2008

(54) Compounds with Atypical Antipsychotic Activity

(75) Inventors: Patrizia Minetti, Rome (IT); Maria Assunta Di Cesare, Rome (IT); Giuseppe Campiani, Chianciano Terme (SI) (IT); Stefania Butini, Siena (IT)

Correspondence Address: NIXON & VANDERHYE, PC, 901 NORTH GLEBE ROAD, 11TH FLOOR, ARLINGTON, VA, 22203, US

(73) Assignee: SIGMA-TAU INDUSTRIE FARMACEUTICHE RIUNITE S.P.A., Rome (IT)

(21) Appl. No.: 11/578,113

(22) PCT Filed: Apr. 5, 2005
(86) PCT No.: PCT/IT05/00182

§ 371 Date: Oct. 10, 2006

(30) Foreign Application Priority Data
Apr. 7, 2004 (IT) RM2004A000178

(51) Int. Cl. A61K 31/554; C07D 513/04; C07D 487/04; A61K 31/55

(52) U.S. Cl. 514/211.1; 540/548; 514/214.01; 540/586

(57) ABSTRACT

Formula (I) compounds with atypical antipsychotic activity

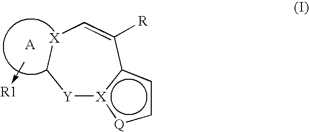

where the meanings of the various groups are as described here below, are endowed with activity characteristic of atypical antipsychotic agents, and therefore are useful as medicaments, particularly for the treatment of schizophrenia, paranoid states, manic-depressive states, affective disorders, social regression, personality regression and hallucinations.

The present invention relates to compounds with atypical antipsychotic activity. In particular, the present invention relates to compounds, the prevalent characteristics of which are the ability to interact with serotonin 5HT₂ₐ receptors and a low affinity for dopamine D₂ receptors, to be used for the preparation of medicaments for the treatment of psychotic disorders such as, for example, schizophrenia.

The present invention also relates to processes for the preparation of said compounds, to their use as medicaments, particularly as atypical antipsychotic medicaments, and to pharmaceutical compositions containing them.

### BACKGROUND TO THE INVENTION

In clinical practice, for the treatment of psychotic disorders including schizophrenia, schizophreniform disorders, schizoaffective disorders, acute and chronic psychotic states related and unrelated to other pharmacological treatments, many compounds with anti-psychotic activity are used. The therapeutic efficacy of such agents is related to their antagonist activity towards dopaminergic D₂ receptors.

These products, which are classified as typical or classic anti-psychotic agents, include phenothiazines (chlorpromazine, pherphenazine, thioridazine, etc.), butyrophenones (aloperidol), thioxanthines (flupenthixol), and substituted benzamides (sulpiride, amisulpride). While, on the one hand, blockade of dopamine D₂ receptors is responsible for the clinical efficacy of these products, on the other it causes the occurrence of severe motor (extrapyramidal syndrome) and neuroendocrine (hyperprolactinaemia) disorders that discourage their use.

Owing to the substantial intensity with which such unwanted effects manifest themselves, the patients undergoing chronic treatment with classic antipsychotic medicaments often abandon the therapy. Over the past decade, products defined as atypical anti-psychotics have come into use, their efficacy being associated with a lower incidence of the above-mentioned side effects.

Better tolerability is not the only advantage offered by the use of atypical antipsychotic agents. Such products prove effective in the treatment of patients refractory to therapy with classic anti-psychotics and also show efficacy against the negative symptoms of schizophrenia and can improve the cognitive function of the schizophrenic patient. A number of atypical antipsychotic agents have, as a common characteristic, a reduced affinity for dopamine D₂ receptors and a greater interaction ability with the serotonin 5-HT₂ₐ receptor. The activity on the serotoninergic system may contribute not only to limiting extrapyramidal-type side effects, but may also enhance their efficacy in the treatment of negative symptoms.

Clozapine is the prototype of this class of drugs. This anti-psychotic agent, which is also effective in the treatment of the negative symptoms of schizophrenia and in schizophrenic patients refractory to other treatments, has, however, given rise to the occurrence of dyscrasia or agranulocytosis in a number of subjects, which means that the patients treated with this antipsychotic medicament must be subjected to strict blood tests. This disadvantageous aspect of clozapine therapy has prompted the search for new antipsychotic agents optionally characterised by an efficacy comparable to that of clozapine, but with a safer pharmacological profile; in this connection see WO 00/006579 and WO 02/010175.

Another important aspect of the use of atypical antipsychotic agents, which are drugs often used in the chronic treatment of diseases such as schizophrenia, is the reduction of the dosage necessary to obtain a therapeutic response and thus the containment of toxicity and accumulation phenomena.

A still perceived problem, then, is that of finding a class of atypical antipsychotic agents which is free of side effects, or which presents such effects to a substantially reduced extent and is also effective at reduced doses.

### SUMMARY OF THE INVENTION

It has now been found that the compounds described in the present invention are endowed with favourable pharmacological activity as atypical antipsychotic agents. One object of the present invention consists therefore in formula (I) compounds

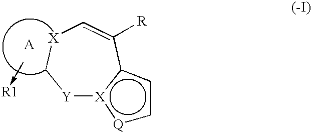

where:

R is C₁-C₄ dialkylamine, where the alkyl groups can be the same or different, 1-piperazinyl optionally substituted in 4 with C₁-C₄ alkyl, said alkyl group optionally substituted with a hydroxyl group, 1-imidazolyl, 1-piperidinyl, optionally substituted in 4 with a C₁-C₄ alkyl group;

R₁ is H or halogen;

Y is CH₂, S;

the two X's can be independently C or N;

A is an aromatic cycle with 5 or 6 carbon atoms;

Q is S, CH═, CH-alkyl(C₁-C₄);

and their pharmaceutically acceptable salts.

Another object of the present invention is a process for the preparation of formula (I) compounds.

Another object of the present invention consists of pharmaceutical compositions containing at least one formula (I) compound in mixtures with pharmaceutically acceptable vehicles and/or excipients.

Another object of the present invention is the use of said compounds as medicaments, particularly for the preparation of medicaments useful for the treatment of psychotic, psychiatric and neurological disorders, particularly disorders related to increased activity of the mesolimbic dopaminergic pathway and/or to mesocortical dopaminergic hypofunctionality, for example, schizophrenia in its positive and negative symptoms, conditions associated with or leading to psychoses, paranoid states, manic-depressive states, affective disorders, or drug-induced psychotic disorders (psychoses in Parkinson's disease).

Said formula (I) compounds are characterised by atypical antipsychotic activity.

The invention will now be described in detail also with the aid of examples.

### DETAILED DESCRIPTION OF THE INVENTION

What is meant by halogen is fluorine, chlorine or bromine.

Pharmaceutically acceptable salts are those salts that do not give rise to unwanted side effects or which are in no way prejudicial to the therapeutic application of the formula (I) compounds.

A first group of preferred compounds is that in which Y is CH₂ in the formula (I) compounds.

A second group of preferred compounds is that in which Y is S in the formula (I) compounds.

Preferred compounds according to the present invention are:
11-(4-methylpiperazin-1-yl)-5H-pyrrolo[1,2-b][2]benzazepine (ST2329);
4-(4-methylpiperazin-1-yl)pyrrolo[2,1-b]thieno[3,2-f][1,3]thiazepine (ST2549);
8-chloro-11-(4-methylpiperazin-1-yl)-5H-pyrrolo[1,2-b][2]benzazepine (ST2776);
3-methyl-11-(4-methylpiperazin-1-yl)-5H-pyrrolo[1,2-b][2]benzazepine (ST2810).

The compounds can be prepared according to the syntheses described in Schemes 1 and 2, respectively, here below.

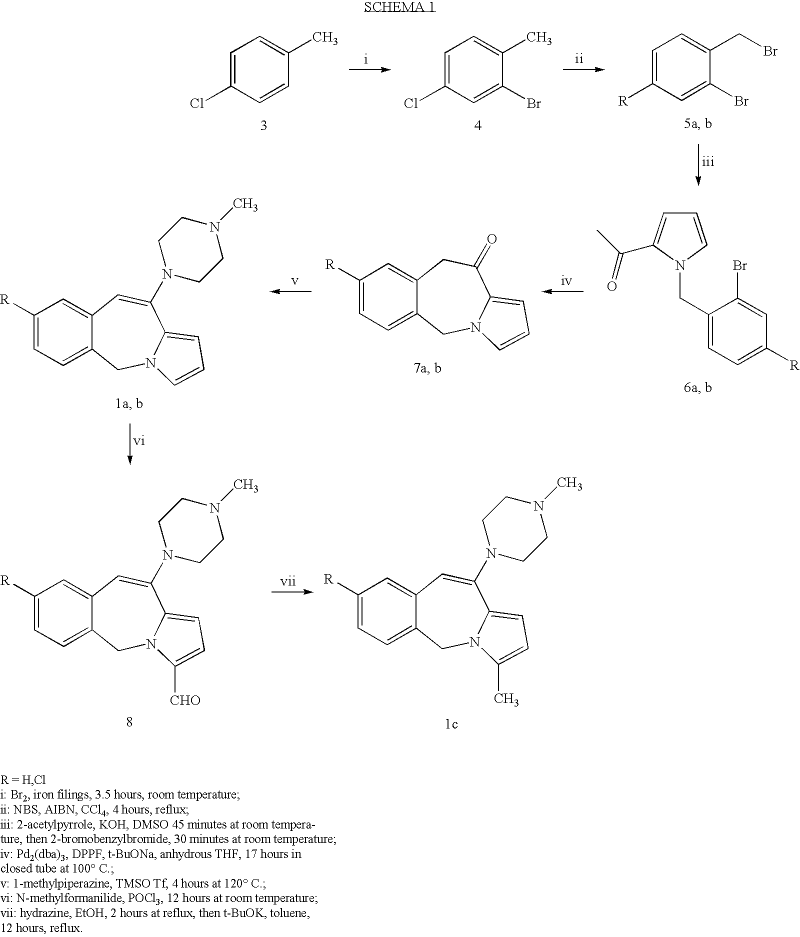

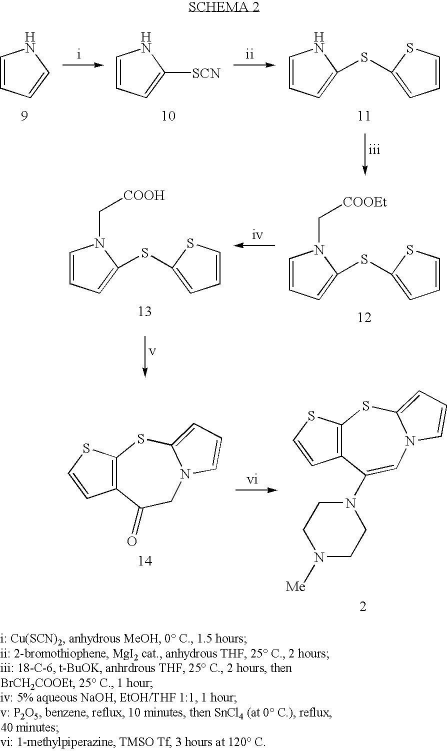

### Description of the Synthesis of 1a-c Derivatives (Scheme 1).

p-Chlorotoluene 3 was subjected to a bromination reaction of the aromatic ring to obtain derivative 4, which was transformed to the corresponding benzylbromide 5b through a radical bromination reaction.

2-Acetylpyrrole in potash (KOH) and dimethylsulphoxide (DMSO) after addition of the appropriate benzylbromide yielded the derivatives 6a,b. The 6a,b compounds were cyclised using a palladium-catalysed reaction, by treatment with tris(dibenzylidene-acetone)dipalladium(0) [Pd₂(dba)₃] and 1,1′-bis(diphenyl-phosphino)ferrocene (DPPF) in the presence of sodium tert-butylate (t-BuONa), obtaining the cyclic ketone derivatives 7a,b. The latter were subsequently treated with trimethylsilyltriflate (TMSOTf) and 1-methylpiperazine to give the enamines 1a and 1b. The 1a derivative was then formylated at position 3 of the heterocyclic system to obtain compound 8, which was reduced, yielding methyl derivative 1c.

### Description of the Synthesis of Derivative 2 (Scheme 2).

Freshly distilled pyrrole 9 was reacted with cupric thiocyanate [Cu(SCN)₂] (prepared from cupric sulphate and sodium thiocyanate) to obtain derivative 10, which, when subjected to a reaction with Grignard salt, yielded thioether 11. Subsequently, thioether 11 was alkylated on pyrrolic nitrogen obtaining ester 12, which was transformed by basic hydrolysis into the corresponding acid 13. Cyclisation in the beta position of the thiophene was accomplished by reacting acid 14 with phosphorus pentoxide (P₂O₅), or with phosphorus pentachloride (PCl₅) and tin tetrachloride (SnCl₄), according to Friedel-Craft conditions. Finally, the cyclic derivative 14 was treated with TMSOTf and then with 1-methylpiperazine to give enamine 2.

It is perfectly evident to any technician with normal experience in the field that the synthesis schemes described above can cover all the variants envisaged in the general formula (I), by simply choosing suitable starting compounds and making any appropriate changes, as necessary, to the reactions illustrated above.

A further object of the present invention are pharmaceutical compositions containing at least one formula (I) compound in mixtures with at least one pharmaceutically acceptable excipient and/or diluent and optionally with additional active ingredients useful in the treatment of psychotic disorders. Examples of such additional active ingredients are phenothiazines, thioxanthenes, butyrophenones, dibenzoxazepines, Rauwolfia alkaloids, and others known to experts in the field.

The formula (I) compounds can be formulated in solid, liquid or semisolid pharmaceutical forms. Examples of liquid formulations are injectable solutions or solutions for oral use, syrups, elixirs, suspensions and emulsions. Examples of solid forms are tablets, capsules, microcapsules, powders, and granulates.

The formula (I) compounds are endowed with antipsychotic activity. This allows their use in the treatment of neuropsychiatric disorders (including, but not limited to, schizophrenia, conditions associated with or giving rise to psychoses, paranoid states, manic-depressive states, affective disorders, drug-induced psychotic disorders (psychoses in Parkinson's disease)). Moreover, further indications may be behavioural disorders in the context of dementias, anxiety manifestations in the elderly, motor disorders induced by drugs (dyskinesia in Parkinson's disease), analgesia and/or anaesthesia.

As demonstrated in the experimental part, the “atypicity” characteristics of the formula (I) compounds make it possible to treat the above-mentioned diseases effectively, at the same time minimising the side effects (extrapyramidal and neuroendocrine disorders) caused by classic antipsychotic agents. The high degree of efficacy, as antipsychotic agents, encountered for these products in animal models, suggests a significant reduction in the dosage necessary to obtain a therapeutic response and thus the containment of toxicity and accumulation phenomena. The reduction of the daily dose to be administered to the patient is a particularly promising aspect in the treatment of chronic diseases such as schizophrenia, which require prolonged exposure to the drug.

The formula (I) compounds can be administered in doses ranging from 0.01 mg/kg to 10 mg/kg, depending upon the severity of the disease to be treated and on its acute or chronic component. Changes in relation to the dosage range indicated are, however, possible in particular conditions, under medical monitoring.

The following examples further illustrate the invention.

### EXAMPLE 1

### Scheme 1

2-Bromo-4-chlorotoluene (4). To a flask containing p-chlorotoluene (10.00 g, 79.00 mmol) and iron filings (4.41 g, 79.00 mmol) was added molecular bromine (5.06 mL, 98.73 mmol) via a drip funnel; the reaction was left to stir for 3.5 hours, after which the mixture was filtered and the product purified by distillation (b.p. 80° C. at 2 mm Hg). 6.71 g of bromoderivative (4) were obtained as a transparent oil with a yield of 40%.

¹H NMR (CDCl₃) (read.);

MS m/z 206 (100, M⁺+H), 169, 125, 99, 89, 73;

Anal. (C₇H₆BrCl) compliant with the expected structure.

2-Bromo-4-chlorobenzylbromide (5b). To the bromoderivative (6.71 g, 32.68 mmol) (4) dissolved in CCl₄ were added N-bromosuccinimide (NBS) (5.82 g, 32.68 mmol), α,α′-azoisobutyronitrile (AIBN) (107.2 mg, 0.65 mmol) and after heating at reflux for 4 hours, the reaction mixture was cooled and filtered on Gooch. The filtrate was washed with aqueous sodium thiosulphate solution and then the organic phase was anhydrified on anhydrous sodium sulphate and the solvent evaporated. The crude reaction product was purified by flash chromatography, using n-hexane as the eluent.

6.00 g of product (5b) are thus obtained as a colourless oil with a yield of 64%.

¹H NMR (CDCl₃) δ 7.58 (d, 1H, J=1.75 Hz), 7.38 (d, 1H, J=8.51 Hz), 7.28 (m, 1H), 4.55 (s, 2H);

MS m/z 284 (M⁺), 205 (100), 169, 124, 89;

Anal. (C₇H₅Br₂Cl) C, H, N compliant with the expected structure.

1-(2-Bromobenzyl)-2-acetylpyrrole (6a). A solution of 2.5 g of KOH in 20 mL of DMSO was stirred for 30 minutes at room temperature. 2-Acetylpyrrole (1.2 g, 10.99 mmol) was then added and the resulting mixture was stirred for another 45 minutes at room temperature, after which the 2-bromobenzylbromide (5a) (3.7 g, 14.8 mmol) was added all at one time and the solution was stirred for another 30 minutes again at room temperature. A saturated sodium chloride solution was then added and the aqueous phase was extracted three times with ethyl acetate. The pooled organic extracts were anhydrified on anhydrous sodium sulphate, filtered and brought to dryness. The crude reaction mixture was purified by flash chromatography (25% ethyl acetate in n-hexane) to give the desired product (6a) with a quantitative yield in the form of a white solid.

¹H NMR (CDCl₃) δ 7.55-7.51 (d, 1H, J=7.4 Hz), 7.14-7.02 (m, 3H), 6.85 (m, 1H), 6.53 (m, 1H), 6.20 (m, 1H), 5.63 (s, 2H), 2.40 (s, 3H);

MS m/z 278 (M⁺), 262, 236, 198 (100), 183, 156, 89;

Anal. (C₁₃H₁₂BrNO)C, H, N compliant with the expected structure.

M.P. 80-81° C.

1-(2-Bromo-4-chlorobenzyl)-2-acetylpyrrole (6b). A solution of 608.2 mg of KOH in 6 mL of DMSO was stirred for 30 minutes at room temperature. 2-Acetylpyrrole (295.7 mg, 2.71 mmol) was then added and the resulting mixture was stirred for another 45 minutes at room temperature, after which 2-bromo-4-chlorobenzylbromide (5b) (1.00 g, 3.52 mmol) was added and the solution was stirred for another 30 minutes, again at room temperature. A saturated sodium chloride solution was then added and the aqueous phase was extracted three times with ethyl acetate. The pooled organic extracts were anhydrified on anhydrous sodium sulphate, filtered and brought to dryness. The crude reaction product was purified by flash chromatography (25% ethyl acetate in n-hexane) to give the desired product (6b), with a quantitative yield, in the form of a white solid.

¹H NMR (CDCl₃) δ 7.55 (d, 1H, J=1.82 Hz), 7.12 (dd, 1H, J=8.65, 1.75), 7.03 (m, 1H), 6.87 (m, 1H), 6.43 (m, 1H), 6.22 (m, 1H), 5.57 (s, 2H), 2.40 (s, 3H);

ES/MS m/z 313 (M⁺+H), 270, 232 (100), 205, 154, 124;

Anal. (C₁₄H₁₁ClBrNO)C, H, N compliant with the expected structure.

M.P. 76-77° C.

11-Oxo-10,11-dihydro-5H-pyrrolo[2,1-b][2]benzazepine (7a). To a solution of Pd₂(dba)₃ (49.4 mg, 0.054 mmol) and DPPF (36.036 mg, 0.065 mmol) in 10 mL of anhydrous tetrahydrofurane were added sodium tert-butylate (152 mg, 1.582 mmol) and 1-(2-bromobenzyl)-2-acetylpyrrole (6a) (200 mg, 0.719 mmol). The reaction mixture was placed in a closed tube, in an argon atmosphere, at 100° C. overnight under vigorous shaking. The mixture was then diluted with ethyl ether and treated with a 0.5 M citric acid solution. The aqueous phase was extracted three times with ethyl ether. The pooled organic extracts were anhydrified on anhydrous sodium sulphate, filtered and brought to dryness. The crude reaction product was purified by flash chromatography (25% ethyl acetate in n-hexane) to give the desired product (7a), with a yield of 65%, in the form of a brown solid.

¹H NMR (CDCl₃) δ 7.31-7.17 (m, 4H), 7.09 (m, 1H), 6.89 (m, 1H), 6.15 (m, 1H), 5.22 (s, 2H), 4.06 (s, 2H);

MS m/z 197 (100, M⁺), 168, 104, 78;

Anal. (C₁₃H₁₁NO)C, H, N compliant with the expected structure.

8-Chloro-11-oxo-10,11-dihydro-5H-pyrrolo[2,1-b][2]-benzazepine (7b). To a solution of Pd₂(dba)₃ (65.99 mg, 0.072 mmol) and DPPF (30.40 mg, 0.054 mmol) in 20 mL of anhydrous tetrahydrofurane were added sodium tert-butylate (203.19 mg, 2.11 mmol) and 1-(2-bromo-4-chlorobenzyl)-2-acetylpyrrol (6b) (300.0 mg, 0.961 mmol). The reaction mixture was placed in a closed tube, in an argon atmosphere, at 100° C. overnight, under vigorous shaking. The mixture was then diluted with ethyl ether and treated with a 0.5 M citric acid solution. The aqueous phase was extracted three times with ethyl ether. The pooled organic extracts were anhydrified on anhydrous sodium sulphate, filtered and brought to dryness. The crude reaction product was purified by flash chromatography (25% ethyl acetate in n-hexane to give the desired product (7b), with a 30% yield, in the form opf a white solid.

¹H NMR (CDCl₃) δ 7.26 (m, 3H), 7.10 (m, 1H), 6.91 (m, 1H), 6.14 (m, 1H), 5.19 (s, 2H), 4.02 (s, 2H); ES/MS m/z 484 (100, 2M⁺+Na), 462 (2M⁺+H), 254 (M⁺+Na), 232 (M⁺+H), 198;

Anal. (C₁₃H₁₀ClNO)C, H, N compliant with the expected structure.

M.P. 189-191° C.

### 11-(4-Methylpiperazin-1-yl)-5H-pyrrolo[2,1-b][2]benzazepine (1a) ST2329

To a solution of (7a) (0.50 g, 2.538 mmol) in 1.266 mL of N-methylpiperazine (1.144 g, 11.421 mmol) were added 1.263 mL of trimethylsilyltriflate (1.551 g, 6.979 mmol) slowly at 0° C. The reaction mixture was placed under argon at 120° C. for 4 hours, and then overnight at room temperature, after which water was added and the mixture extracted with dichloromethane. The pooled organic extracts were anhydrified on anhydrous sodium sulphate, filtered and brought to dryness. The crude reaction product was purified by chromatography on allumina (25% tetrahydrofurane in n-hexane) to give the desired product, with a 60% yield, in the form of a clear solid.

¹H NMR (CDCl₃) δ 7.21-7.06 (m, 4H), 6.68 (m, 1H), 6.26 (m, 1H), 6.14 (m, 1H), 5.91 (s, 1H), 4.83 (s, 2H), 3.17-3.12 (m, 4H), 2.59-2.47 (m, 4H), 2.36 (s, 3H);

MS m/z 279 (M⁺), 256, 209 (100), 180, 168, 152;

Anal. (C₁₈H₂₁N₃) C, H, N compliant with the expected structure.

M.P. 137-138° C.

### 8-Chloro-11-(4-methylpiperazin-1-yl)-5H-pyrrolo[2,1-b][2]benzazepine (1b) ST 2776

To a solution of (7b) (0.045 g, 0.194 mmol) in 100 μL of N-methylpiperazine (87.45 mg, 0-873 mmol) were added 96.54 μL of trimethylsilyltriflate (118.55 mg, 0.533 mmol) slowly at 0° C. The reaction mixture was placed under argon at 120° C. for 4 hours, and then overnight at room temperature. Water was then added and the mixture was extracted with dichloromethane. The pooled organic extracts were anhydrified on anhydrous sodium sulphate, filtered and brought to dryness. The crude reaction product was purified by chromatography on allumina (25% tetrahydrofurane in n-hexane) to give the desired product, with a 50% yield, in the form of a clear solid.

¹H NMR (CDCl₃) δ 7.13 (m, 3H), 6.69 (m, 1H), 6.29 (m, 1H), 6.17 (m, 1H), 5.80 (s, 1H), 4.80 (s, 2H), 3.15 (m, 4H), 2.55 (m, 4H), 2.36 (s, 3H); ES/MS m/z 314 (100, M⁺+H), 280, 216; Anal. (C₁₈H₂₀ClN₃) C, H, N compliant with the expected structure.

M.P. 145-147° C.

### 3-Formyl-11-(4-methylpiperazin-1-yl)-5H-pyrrolo[2,1-b][2]benzazepine (8)

Derivative (1a) (200.0 mg, 0.717 mmol) was dissolved in 500.0 μL of N-methylformanilide and added with 194.1 μL of the formylating complex prepared by reacting 110.60 μL of N-methylformanilide (121.11 mg, 0.896 mmol) and 83.52 μL of phosphorus oxychloride (POCl₃) (137.4 mg, 0.896 mmol) for 30 minutes at room temperature. The reaction mixture was left to stir for 12 hours at room temperature. Water was then added and extraction done with dichloromethane. The organic extracts were anhydrifed on anhydrous sodium sulphate and evaporated. The crude product was purified by flash chromatography (20% methanol in ethyl acetate) obtaining the formylated derivative (8), with a 25% yield, as a yellow oil.

¹H NMR (CDCl₃) δ 9.48 (s, 1H), 7.46 (m, 1H), 7.20 (m, 3H), 6.85 (d, 1H, J=4.01 Hz), 6.34 (d, 1H, J=4.50 Hz), 6.21 (s, 1H), 5.52 (br s, 2H), 3.11 (m, 4H), 2.59 (m, 4H), 2.37 (s, 3H);

ES/MS m/z 308 (100, M⁺+H), 280;

Anal. (C₁₉H₂₁N₃O)C, H, N compliant with the expected structure.

### 3-Methyl-11-(4-methylpiperazin-1-yl)-5H-pyrrolo[2,1-b][2]benzazepine (1c) ST 2810

To a solution of formylderivative (8) (110.0 mg, 0.358 mmol) in absolute ethanol (2.3 mL) was added hydrated hydrazine (390.60 μL, 12.54 mmol) and the reaction mixture was left to react at reflux for 2 hours. The ethanol was evaporated and the residue reacted with potassium tert-butylate (t-BuOK) (120.52 mg, 1.074 mmol) in toluene (2.5 mL) at reflux for 12 hours. The cooled reaction mixture was taken up with water and extraction done with dichloromethane. The pooled organic phases were anhydrified on anhydrous sodium sulphate, filtered and the solvent evaporated. The crude product was purified by chromatography on allumina (20% tetrahydrofurane in n-hexane) to give the product (1c), with a 30% yield, as a yellow oil.

¹H NMR (CDCl₃) δ 7.16 (m, 4H), 6.16 (d, 1H, J=3.79 Hz), 5.91 (m, 2H), 4.75 (s, 2H), 3.15 (m, 4H), 2.55 (m, 4H), 2.35 (s, 6H); ES/MS m/z 294 (100, M⁺+H), 194; Anal. (C₁₉H₂₃N₃) C, H, N compliant with the expected structure.

### EXAMPLE 2

### Scheme 2

### 2-Thiocyanopyrrole (10)

A solution of pyrrole (2.0 g, 29.8 mmol) distilled fresh was solubilised in 25 mL of anhydrous methanol under argon. To the solution, cooled to 0° C., was added Cu(SCN)₂ (10.7 g, 59.7 mmol) (prepared from CuSO₄ and NaSCN) and the mixture was left to react under magnetic stirring for 1.5 hours. The reaction mixture was filtered on Gooch and the filtrate poured into a beaker containing ice and sodium chloride. After extraction of the aqueous mixture with ethyl acetate, the pooled organic extracts were anhydrified on anhydrous sodium sulphate and the solvent evaporated. 3.2 g of crude product were obtained, which was purified by flash chromatography (30% n-hexane in ethyl acetate), to give 1.5 g of 10 as an orange oil with a yield of 41%.

¹H NMR (CDCl₃) δ 8.80 (br s, 1H), 6.98 (m, 1H), 6.64 (m, 1H), 6.27 (m, 1H);

Anal. (C₅H₄N₂S)C, H, N compliant with the expected structure.

### 2-(2-Thienylsulphanyl)-1H-pyrrole (11)

To an anhydrous three-necked flask into which anhydrous metallic magnesium (1.4 g, 57.6 mmol) was introduced, covered with a film of anhydrous tetrahydrofurane, were added 3-4 drops of a solution of 2-bromothiophene in anhydrous tetrahydrofurane and one crystal of 12. Once the formation of the Grignard salt was started, addition of the solution of 2-bromothiophene (7.1 g, 43.5 mmol) in anhydrous tetrahydrofurane (40 mL) was completed and the resulting mixture was heated at reflux for 0.5 hours, after which, via a drip funnel, a solution of 10 (3.6 g, 29.0 mmol) in anhydrous tetrahydrofurane (100 mL) was added to the Grignard salt, at room temperature, and left to react at room temperature for 2 hours. After this time, a saturated solution of ammonium chloride was added and the aqueous phase was extracted with diethylether. The ethereal phase was anhydrified on anhydrous sodium sulphate and the solvent evaporated. The reaction was purified on a flash chromatography column (50% n-hexane in dichloromethane) to give 3.8 g of thioether (11), with a 72% yield, as a brown oil.

¹H NMR (CDCl₃) δ 8.29 (br s, 1H), 7.25 (d, 1H, J=6.2 Hz), 7.07 (d, 1H, J=3.9 Hz), 6.92 (m, 1H), 6.83 (m, 1H), 6.53 (m, 1H), 6.23 (m, 1H);

MS m/z 181 (100, M⁺), 153, 115, 98, 84, 71;

Anal. (C₈H₇NS₂) C, H, N compliant with the expected structure.

### Ethyl-2-[2-(2-thienylsulphanyl)-1H-1-pyrrolyl]acetate (12)

To a suspension of 18-crown-6 (54.0 mg, 0.203 mmol) and t-BuOK (456.6 mg, 4.069 mmol) in anhydrous tetrahydrofurane (10 mL) was added a solution of (11) (570.0 mg, 3.13 mmol) in anhydrous tetrahydrofurane (10 mL) and the mixture was left to react for 2 hours under argon at room temperature. A solution of ethyl bromoacetate (1.04 g, 6.26 mmol) in anhydrous tetrahydrofurane (6 mL) was then added to the reaction mixture and the mixture was reacted for 1 hour at room temperature. 10 mL of water were added and the tetrahydrofurane was evaporated. The aqueous residue was extracted with ethyl acetate and the organic phase anhydrified and evaporated, obtaining 1.4 g of crude product which was purified by flash chromatography (50% n-hexane in dichloromethane) to give 674.0 mg of product 12 as a pink oil with a yield of 80%.

¹H NMR (CDCl₃) δ 7.21 (d, 1H, J=4.9 Hz), 6.97 (d, 1H, J=3.8 Hz), 6.89-6.83 (m, 2H), 6.60 (m, 1H), 6.22 (m, 1H), 4.79 (s, 2H), 4.12 (q, 2H, J=14.5, J=7.0 Hz), 1.22 (t, 3H, J=7.3 Hz);

MS m/z 267 (100, M⁺), 238, 221, 193, 180, 97;

Anal. (C₁₂H₁₃NO₂S₂) C, H, N compliant with the expected structure.

### 2-[2-(2-thienylsulphanyl)-1H-1-pyrrolyl]acetic acid (13)

The ethyl ester (12) (580.0 mg, 2.17 mmol) was dissolved in a mixture of ethanol/tetrahydrofurane (1:1, 8 mL), added with an aqueous solution of 5% soda (6 mL) and left to react at room temperature for 1 hour under stirring. The solution was acidified with hydrochloric acid 1N, the ethanol and tetrahydrofurane were evaporated and the aqueous mixture extracted with ethyl acetate. The pooled organic extracts were anhydrified on anhydrous sodium sulphate and the solvent removed in vacuo. 510 mg of acid (13) were obtained as a white oil. Yield: 99.9%.

¹H NMR (CDCl₃) δ 9.34 (br s, 1H), 7.0 (d, 1H, J=4.6 Hz), 6.98 (d, 1H, J=4.2 Hz), 6.85 (m, 1H), 6.76 (m, 1H), 6.59 (m, 1H), 6.20, (m, 1H), 4.77 (s, 2H);

Anal. (C₁₀H₉NO₂S₂) C, H, N compliant with the expected structure.

### 4,5-Dihydropyrrolo[2,1-b]thieno[3,2-f][1,3]thiazepin-4-one (14)

METHOD A. To a solution of acid (13) (150.0 mg, 0.627 mmol) in anhydrous toluene (2 mL) was added anhydrous P₂O₅ (200.0 mg, 1.255 mmol) and the suspension was stirred at reflux temperature for 5 hours under argon. The reaction mixture was cooled and filtered, and the filtrate was washed with a saturated sodium chloride solution. The organic phase was separated and anhydrified on anhydrous sodium sulphate and the toluene evaporated. The crude reaction product was purified by flash chromatography (50% n-hexane in dichloromethane) obtaining the cyclic product (14) as a yellow oil with a yield of 10%.

¹H NMR (CDCl₃) δ 7.51 (d, 1H, J=5.8 Hz), 7.10 (d, 1H, J=5.1 Hz), 6.89 (m, 1H), 6.41 (m, 1H), 6.11 (m, 1H), 5.05 (s, 2H);

MS m/z 221 (100, M⁺), 188, 160, 147, 121;

Anal. (C₁₀H₇NOS₂) C, H, N compliant with the expected structure.

METHOD B. A solution of acid (13) (810.0 mg, 3.389 mmol) in anhydrous benzene (12 mL) was added with PCl₅ (850.0 mg, 4.067 mmol) and heated at reflux temperature under argon for 10 minutes. The reaction mixture was cooled to 0° C., added with 500 μL of SnCl₄ (971.0 mg, 3.728 mmol) and placed at reflux temperature for 40 minutes. Ethyl ether (50 mL) was added to the reaction, and the organic phase, washed with hydrochloric acid 1N, was anhydrified on anhydrous sodium sulphate and the solvent evaporated. The purification was done as described for Method A. The cyclic product (14) was obtained with a 7% yield. The physico-chemical data are concordant with those of the product obtained using synthesis Method A.

### 4-(4-Methylpiperazino)pyrrolo[2,1-b]thieno[3,2-f][1,3]thiazepine (2) ST 2549

To the cyclic product (14) (30.0 mg, 0.136 mmol) dissolved in N-methylpiperazine (500 μL) were added 70 μL of trimethylsilyltriflate (82.97 mg, 0.373 mmol) and the mixture was left to react at 120° C. for 3 hours under argon and then overnight at room temperature. The reaction mixture added with water was extracted with dichloromethane. The pooled organic extracts were anhydrified and evaporated. The crude reaction product was purified on a flash chromatography column (20% methanol in ethyl acetate) to give 35 mg of final product (2) as a yellow oil with a yield of 85%.

¹H NMR (CDCl₃) δ 7.16 (d, 1H, J=5.8 Hz), 7.03 (d, 1H, J=5.7 Hz), 6.71 (m, 1H), 6.53 (s, 1H), 6.20 (m, 1H), 6.04 (m, 1H), 2.94 (m, 4H), 2.51 (m, 4H), 2.35 (s, 3H);

ES/MS m/z 304 (100, M⁺+H);

Anal. (C₁₅H₁₇N₃S₂), C, H, N compliant with the expected structure.

### In-Vitro Pharmacology

Evaluation of ability to interact with D₁, D₂, D₃ and 5HT₂ₐ, Receptors

Interaction with the D₁, D₂, and 5HT₂ₐ receptors was studied using different cerebral areas (striate D₁ and D₂; prefrontal cortex 5HT₂ₐ) according to the procedure described in the literature (Campiani, et. al.; J. Med. Chem., pp. 3763-3772, 1998); transfected cell membranes were used for the D₃ receptor (cloned dopamine receptor subtype 3, rat, Sf9 cells, Signal Screen). Interaction with the D₁ receptor was evaluated using [³H]-SCH 23390 (0.4 μM) as the radioligand, and the non-specific binding was determined in the presence of (−)-cis-flupenthixol (10 μM). For the D₂ receptor ³H-Spiperone (0.2 nM) was used and the non-specific binding was determined in the presence of 100 μM of (−)sulpiride. As regards the D₃ receptor, the radioligand selected 3H-7-OH-DPAT was used at the concentration of 0.2 μM and the non-specific binding was obtained in the presence of dopamine 10 μM. Lastly, interaction with 5HT₂ₐ was evaluated using ³H-ketanserin (0.7 μM) and the non-specific binding was determined in the presence of methysergide 1 μM.

### Results

Table 1 gives the means and standard errors of the affinity values expressed as Ki (nM) of the study product ST2329 for the dopaminergic receptors D₁, D₂ and D₃, and the serotoninergic receptor 5-HT₂ₐ. In addition, the affinity values for the above-mentioned receptor types are also presented in relation to the atypical antipsychotic agents clozapine and olanzapine and the typical antipsychotic aloperidol.

TABLE 1

| | 5-HT₂ₐ | D₂ | D₁ | D₃ |
|---|---|---|---|---|
| Compound | (Kᵢ: nM) | (Kᵢ: nM) | (Kᵢ: nM) | (Kᵢ: nM) |
| ST2329 | 7.2 | 578 | 125 | 224 |
| Olanzapine | 4 | 69 | 85 | 39 |
| Clozapine | 10 | 250 | 353 | 319 |
| Aloperidol | 164 | 4.8 | 318 | 18 |

The compound ST2329 shows high affinity for the serotonin 5HT₂ₐ receptor and less ability to interact with the dopaminergic receptors investigated. This receptor profile is similar to that shown by the atypical antipsychotic agents (clozapine and olanzapine), which, compared to that of the typical antipsychotics (aloperidol), is characterised by a preferential ability to interact with serotoninergic receptors of the 5-HT₂ₐ type and weak affinity for the D₂-type dopaminergic receptors.

Table 2 shows the inhibition constants (pKi) of the compound ST2329 and of the reference compounds for the D₁, D₂ D₃ and 5HT₂ₐ receptors and the 5HT₂ₐ:D₂ relative affinity ratio.

This latter parameter, if greater than the value 1.12, is regarded as a valid indicator for describing the atypicity profile of an antipsychotic agent (Meltzer, et al.; J. Pharmacol. Exp. Ther., 251 (1) pp 238-245 1989). Moreover, the Log Y parameter is also indicated, which, considering the relative affinity values of each product for the 5HT₂, D₂, and D₁ receptors, identifies and distinguishes a classic anti-psychotic (Log Y>6.48) from an atypical one (Log Y<6.48) (Meltzer, et al.; J. Pharmacol. Exp. Ther., 251 (1) pp 238-245 1989).

TABLE 2

| | D₁ | D₂ | D₃ | 5HT_{2A} | | |
|---|---|---|---|---|---|---|
| Compound | pKi | pKi | pKi | pKi | 5HT₂ₙ/D₂ | Log Y |
| Clozapine | 6.45 | 6.60 | 6.50 | 8.00 | 1.21 | 3.89 |
| Olanzapine | 7.07 | 7.16 | 7.59 | 8.40 | 1.17 | 4.69 |
| Aloperidol | 6.50 | 8.32 | 7.74 | 6.78 | 0.82 | 9.14 |
| ST2329 | 6.90 | 6.24 | 6.65 | 8.14 | 1.30 | 3.20 |

Each of the above-mentioned parameters confirms that the product ST2329 belongs to the pharmacological class of the atypical antipsychotic agents and shows a better atypicity profile than those of the reference compounds. These results predict that the antipsychotic activity of this product may be accompanied by only a limited likelihood of the occurrence of unwanted effects such as the motor (extrapyramidal syndrome) and/or neuroendocrine (induction of hyperprolactinaemia) disorders that occur as a result of the chronic administration of classic antipsychotics (aloperidol) or in the early phases of treatment with a number of atypical antipsychotics (risperidone, olanzapine).

### In-Vivo Pharmacology

A. Evaluation of antipsychotic potency of ST2329. Effect of Treatment in Active Avoidance Test.

The administration of incremental doses of an antipsychotic drug in the rat causes inhibition of the active avoidance response and an increase in the number of escape responses without any increase in failures. This change in the animal's avoidance behaviour is characteristic of the effect of the administration of a compound endowed with anti-psychotic activity. Therefore, the evaluation of such behaviour enables us to detect and determine the antipsychotic capacity of a product.

### EXPERIMENTAL PROCEDURE

Male Fischer 344 rats weighing 180 g (Charles River) were used. The product ST2329 was administered to the animals orally 60 minutes prior to the test. The product ST2329 was administered at doses of 0.25 mg/5 ml/kg, 0.5 mg/5 ml/kg, 1.0 mg/5 ml/kg, 1.5 mg/5 ml/kg, 3 mg/5 ml/kg, and 6 mg/5 ml/kg.

To carry out the test to assess the avoidance behaviour of the rats a piece of equipment was used (Ugo Basile) consisting of a plastic cage divided into two compartments by a partition wall with an aperture in it allowing communication between the two sectors. Each of the two sectors can be illuminated by a 10 Watt lamp placed above a plexiglas lid covering the cage. A programming device allows the regulation of the duration and frequency of the unconditioned and discriminatory stimuli and a computerised system acquires the experimental data. During the study, the discriminatory stimulus (light) was presented 3 seconds before the unconditioned stimulus (0.3 mA electric shock for 4 seconds). In each session, one test per minute was performed making a total of 20 tests a day. One session was con-ducted per day.

The procedure consists in the familiarisation of the animal for 1 minute with the test cage followed by the series of tests, the start of which is signaled by the light stimulus; the discriminatory stimulus is followed a few seconds later by the administration of an electric shock. The rat can avoid the shock by escaping to the adjacent compartment. Responses registered after switching on the light and before the shock interrupt the discriminatory stimulus and are considered “avoidances”; responses registered during the shock period terminate both the discriminatory stimulus and the unconditioned stimulus and are considered “escapes”; inability to avoid the shock is considered “failure”.

Before treatment with the study compounds, the animals were selected in relation to their ability to master the task. Admitted to the test assessing the effect of treatment were those rats that achieved at least 75% of avoidances in baseline tests. The results were expressed as means and standard errors. The number of conditioned responses (avoidances) was used to calculate, by means of non-linear regression implemented using the GraphPad Prism data analysis program, the dose of study product capable of reducing the value of this variable (avoidance response capacity) by 50% (ED₅₀) compared to baseline values.

### Results

The product ST2329 inhibits the avoidance response capacity of the animals in a dose-dependent manner.

The value of the dose capable of reducing the avoidance response capacity by 50% (ED₅₀) is 0.56 mg/kg. This activity of the product ST2329 is better than that determined in the same experimental conditions for the atypical antipsychotic agent clozapine.

As reported in Table 3, the antipsychotic potency of ST2329 is exerted at doses much lower than those needed for a treatment with clozapine.

TABLE 3

| | |
|---|---|
| | Inhibition of avoidance response |
| Compound | (ED₅₀) |

| | | |
|---|---|---|
| ST2329 | mg/kg | 0.56 |
| | μmol/kg | 2.00 |
| Clozapine | mg/kg | 4.90 |
| | μmol/kg | 15.0 |

### B. Evaluation of Atypicity Potential of ST2329. Effect of Treatment in Catalepsy Test.

Particularly significant for verifying the atypicity characteristics of an antipsychotic agent is evaluation of the effects induced by the product in the catalepsy test which shows the type of influence on the D₂ dopaminergic receptors of the nigrostriatal dopaminergic system. The catalepsy test is regarded as the most appropriate animal model for describing the possibility of occurrence of motor disorders (EPS: extrapyramidal syndrome). The atypical antipsychotics subjected to this evaluation manifest a lack of such effects or the presence only of negligible effects at the doses at which they exert their antipsychotic potency.

### Experimental Procedure

The test was carried out on male rats of the Wistar strain (n=7 animals); evaluation of catalepsy was done by means of a metal rod measuring 0.6 cm in diameter positioned at a distance of 10 cm from the work surface. The study substance ST2329 was administered orally at the dose of 100 mg/kg 60 minutes before the evaluation test. Subsequent observations were recorded at 60, 90, 120, 180, 240, and 300 minutes after administration. The test consisted in positioning the animal with its front paws on the bar and in chronometrically measuring the time the animal remained hanging from the bar, considering an endpoint of 120 seconds (N. A. Moore, et al.; Journal of Pharmacology and Experimental Therapeutics, Vol. 262 pp 545-551 (1992)).

### Results

As a result of oral treatment with a dose of ST2329 equal to 100 mg/kg no presence of catalepsy was detected in the animals (ED₅₀=>100 mg/kg). These results confirm the poor ability to interact with the D₂ receptors of striate predicted by the 111-vitro evaluations (affinity for D₂ receptors: 576 nM). The evaluation of the in-vivo atypicity index (ratio of the ED₅₀ in the catalepsy test to the ED₅₀ in the active avoidance test, see Table 4), regarded as a descriptive criterion of the possibility of occurrence of extra-pyramidal effects at the dose at which an anti-psychotic agent exerts its therapeutic efficacy, indirectly suggests that the study product ST2329 is a better atypical antipsychotic than the antipsychotic agent clozapine.

TABLE 4

| | Inhibition of | | Induction of | | |
|---|---|---|---|---|---|
| | avoidance | | catalepsy | | Atypicity |
| Compound | response (ED₅₀) | | (ED₅₀) | | index |
| ST2329 | mg/kg | 0.56 | mg/kg | 100 | 179 |
| | μmol/kg | 2.00 | μmol/kg | 357.9 | |
| Clozapine | mg/kg | 4.90 | mg/kg | 100 | 18.4 |
| | μmol/kg | 15.0 | μmol/kg | 275.6 | |

Overall, the results obtained suggest that the study product ST2329 exerts a preferential effect more on the mesolimbic dopaminergic system than on the mesostriatal system, since the doses necessary to inhibit the avoidance response are considerably less than those needed to induce catalepsy in the animal. This evidence suggests the possibility of using the product in the treatment of psychotic states or to treat the symptoms of schizophrenia that are associated with a condition of hyperactivity of the dopaminergic transmission of the mesolimbic pathways. Therefore, as a result of the influence demonstrated on the dopaminergic pathways investigated, ST2329 can be classified as an atypical antipsychotic agent with only a weak tendency to induce acute and chronic extrapyramidal symptoms at the doses at which it manifests its efficacy as an antipsychotic agent. Moreover, the type of activity detected with the above-mentioned tests, recognised as valid instruments for identifying new atypical antipsychotics, indicates that ST2329 can be used in the therapy of psychotic states and/or schizophrenia at lower dosages than those needed with the atypical antipsychotic clozapine.

1. A compound of formula (I) 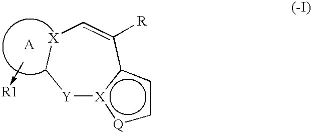
where:
R is C₁-C₄ dialkylamine, where the alkyl groups can be the same or different, 1-piperazinyl optionally substituted in 4 with C₁-C₄ alkyl, said alkyl group optionally substituted with a hydroxyl group; 1-imidazolyl, 1-piperidinyl, optionally substituted in 4 with a C₁-C₄ alkyl group;
R₁ is H or halogen;
Y is CH₂, S;
the two X's can be independently C or N;
A is an aromatic cycle with 5 or 6 carbon atoms;
Q is S, CH═, CH-alkyl(C₁-C₄);
and their pharmaceutically acceptable salts.

2. A compound according to claim 1, wherein Y is CH₂.

3. A compound according to claim 1, wherein Y is S.

4. A compound according to claim 1, selected from the group consisting of
11-(4-methylpiperazin-1-yl)-5H-pyrrolo[1,2-b][2]benzazepine;
4-(4-methylpiperazin-1-yl)pyrrolo[2,1-b]thieno[3,2-f][1,3]thiazepine;
8-chloro-11-(4-methylpiperazin-1-yl)-5H-pyrrolo[1,2-b][2]benzazepine;
3-methyl-11-(4-methylpiperazin-1-yl)-5H-pyrrolo[1,2-b][2]benzazepin.

5. Pharmaceutical compositions containing at least one compound according to claim 1 in mixtures with pharmaceutically acceptable vehicles and/or excipients.

6. Compositions according to claim 5, which contain additional active ingredients useful in the treatment of psychotic disorders.

7. Compositions according to claim 5, in which said additional active ingredients are selected from the group consisting of pheno-thiazines, thioxanthenes, butyrophenones, dibenzoxazepines, and Rauwolfia alkaloids.

8. Use of compounds according to claim 1 as medicaments.

9. Use of compounds according to claim 1 for the preparation of a medicament with antipsychotic activity.

10. Use according to claim 9, in which said medicament is useful for the treatment of psychotic, psychiatric and neurological disorders, particularly disorders related to increased activity of the mesolimbic dopaminergic pathway and/or to mesocortical dopaminergic hypofunctionality.

11. Use according to claim 10, in which said disease is selected from the group consisting of schizophrenia with its positive and negative symptoms, conditions associated with or giving rise to psychoses, paranoid states, manic-depressive states, affective disorders, and drug-induced psychotic disorders (psychoses in Parkinson's disease).

12. Use according to claim 10, in which said disease is selected from the group consisting of behavioural disorders in the context of dementias, anxiety manifestations in the elderly, motor disorders induced by drugs (dyskinesia in Parkinson's disease), analgesia and/or anaesthesia.

13. Use according to claim 9, in which said medicament prevalently possesses the ability to interact with the 5HT₂ₐ receptor and has low affinity for the D₂ receptor.
